(19) United States
(12) Patent Application Publication
(10) Pub. No.: US 2011/0016580 A1
(43) Pub. Date: Jan. 20, 2011

(54) Cell Division and Proliferation Preferred Regulatory Elements and Uses Thereof

(75) Inventors: Xiping Niu, Johnston, IA (US); Nicholas Bate, Urbandale, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL INC., Johnston, IA (US)

(21) Appl. No.: 12/428,177

(22) Filed: Apr. 22, 2009

Related U.S. Application Data
(63) Continuation of application No. 11/751,411, filed on May 21, 2007, now abandoned.
Application No. 11/751,411 is a continuation of application No. 10/388,359, filed on Mar. 13, 2003, now abandoned.
(60) Provisional application No. 60/364,062, filed on Mar. 13, 2002.

(51) Int. Cl. C12N 15/82; C07H 21/04; C12N 15/63; A01H 5/00; A01H 5/10; C12N 5/10

(52) U.S. Cl. 800/278; 536/24.1; 435/320.1; 800/298; 800/320.1; 800/320.3; 800/320.2; 800/320; 435/419; 435/412

(57) ABSTRACT

The present invention provides compositions and methods for regulating expression of nucleotide sequences in a plant. The compositions are novel nucleic acid sequences which confer cellular division and/or proliferation-preferred regulation of operably attached nucleotide sequences. Methods for expressing an isolated nucleotide sequence in a plant using the regulatory sequences, expression cassettes, vectors and resultant plants are also provided.

### CROSS-REFERENCE TO RELATED APPLICATIONS

This application is a continuation of non-provisional U.S. application Ser. No. 11/751,411 filed May 21, 2007, which is a continuation of non-provisional U.S. application Ser. No. 10/388,359 filed Mar. 13, 2003, now abandoned, which claims priority to provisional U.S. Application Ser. No. 60/364,062 filed Mar. 13, 2002, all of which applications are hereby incorporated by reference.

### FIELD OF THE INVENTION

The present invention relates to the field of plant molecular biology, more particularly to regulation of gene expression in plants.

### BACKGROUND OF THE INVENTION

Expression of isolated DNA sequences in a plant host is dependent upon the presence of operably linked regulatory elements that are functional within the plant host. Choice of the regulatory sequences will determine expression of the isolated DNA sequences within the host. Where continuous expression is desired throughout the cells of a plant, constitutive promoters are utilized. In contrast, where gene expression in response to a stimulus is desired, inducible promoters are the regulatory element of choice. Where temporal or spatial expression is desired tissue-preferred or developmentally specific promoters and/or terminators are used. These regulatory elements can drive expression in specific tissues or organs or during a specific developmental time period. Additional regulatory sequences upstream and/or downstream from the core sequences can be included in expression cassettes of transformation vectors to bring about varying levels of expression of isolated nucleotide sequences in a transgenic plant.

Proliferating cell nuclear antigen (PCNA) is an auxiliary protein of DNA polymerase δ and it is highly conserved among eukaryotes. Stimulation of growth of quiescent plant cells by phytohormones, such as auxins and cytokinins, leads to the entry of cells into the G1 or S phase of the cell cycle from the G0 phase. Several genes or cDNAs for mRNAs that are expressed during the G1 (G0)-S phase transition or during the S phase of the cell cycle have been isolated and studied with respect to gene expression. Among the proteins associated with DNA synthesis, PCNA is known as a auxiliary protein of DNA polymerase δ and it is one of the factors that is essential for the synthesis of the leading strand during replication in vitro of simian virus 40 DNA. In addition, PCNA is also required for DNA-excision repair. The gene for PCNA is highly conserved among eukaryotes, including higher plants. A plant gene for PCNA was first isolated from Rice and the rice PCNA gene was approximately 62% identical for that of rat PCNA.

The expression of PCNA is correlated to the proliferative state of cells; in mammalian cells the amount of PCNA is very low in quiescent cells and increases dramatically when the cells are stimulated to proliferate. Thus the temporal and spatial expression of the PCNA gene and its regulatory elements provide a unique opportunity to direct expression to actively dividing cells. Isolation and characterization of cell division and cell proliferation-preferred promoters and terminators that can serve as regulatory elements for expression of isolated nucleotide sequences of interest in actively dividing cells, is needed for improving yield and health of plants. For example, regulatory elements directed to cell proliferation would be valuable allowing for the manipulation of growth of plants to provide critical nutrients to cells which are currently undergoing cell division, to provide markers of expression so that critical developmental periods may be identified to improve overall plant health or to manipulate the development of organs, flowering or other states associated with the proliferation of plant cells. As can be seen from the foregoing, there is a continuing need in the art for providing for temporal and spatial regulation of DNA sequences for cell proliferation, organ development and the like.

It is thus an object of the present invention to provide novel regulatory elements which provide for cell division and or cell proliferation specific expression of operably linked DNA sequences for improvement in health, productivity and yield of plants.

A further object is to provide a mechanism for manipulating cellular proliferation and concomitant organ development to achieve increased yield, to control inflorescence number, arrangement or other reproductive development, to identify stages of organ development, etc. in plants.

Still another object of the invention is to provide for temporal and spatial regulation of DNA sequences specific to tissues and organs of the plant with actively dividing cells.

It is yet another object of the invention to provide for regulation of DNA sequences with tissue preference of the immature ear and early kernel tissue of maize.

Finally, it is an object of the present invention to provide genetic material which can used to screen other genomes to identify other regulatory elements with similar effects from other plant sources or even from animal sources.

Other objects of the invention will become apparent from the description of the invention which follows.

### SUMMARY OF THE INVENTION

According to the invention there is provided herein a regulatory element isolated from maize which comprises the following: one or more Tb1/PCF binding sites (GGACCC), a TATA box, and is capable of driving expression of linked genes consistent with a PCNA2 expression pattern in plant cells. Preferably the regulatory element will have approximately 65% homology to SEQ ID NO: 1, or hybridize under conditions of high stringency to this sequence, or sequences from SEQ ID NO: 2. The invention also comprises expression constructs comprising the regulatory elements of the invention operably linked to DNA sequences, vectors incorporating said expression constructs, plant cells transformed with these constructs and resultant plants regenerated from or descended from the same. The regulatory elements of the invention provide for expression of operably linked sequences in actively dividing tissues and also provides for tissue preferred expression in the immature ear and early kernel tissue of maize.

### BRIEF DESCRIPTION OF THE DRAWINGS

FIG. 1 is a Northern analysis of the maize PCNA2 gene expression in wild type plant (W22), W22 plant introgressed with the teosinate 1L chromosome (T1L), W22 plant introgressed with the teosinate 1L and 3L chromosomes (T1L3L), Tb1/tb1-mum3 heterozygote (het) and tb1-mum3 homozygote (tb1). All are in W22 background.

FIG. 2 is a diagram showing the PHP 18978 plasmid incorporating the PCNA2 regulatory element of the invention.

FIG. 3 is the sequence (SEQ ID NO: 2) of the PHP plasmid depicted in FIG. 2.

FIG. 4 is alignment of maize PCNA2 (SEQ ID NO: 1) and rice PCNA (SEQ ID NO: 3) promoter regions. The identical sequences between maize PCNA2 (ZmPCNA2) and rice PCNA (OsPCNA) promoters are in shade and gaps are represented by hyphens. The Tbl/PCF binding sites are in bold and indicated by stars. TATA box (TATA) and the first codon (Met) for coding regions are also indicated under the sequences.

FIG. 5 is a model for Tbl/PCFs regulated PCNA2 gene expression. Tbl and PCFs compete for the same binding sites in PCNA2 gene promoter. Under normal condition, Tbl occupies the binding sites; PCNA2 expression is blocked or reduced. When PCFs occupy the binding sites, PCNA2 gene expression is activated.

### DETAILED DESCRIPTION OF THE INVENTION

PCNA (Proliferating Cell Nuclear Antigen) plays an important role in the cell cycle, as well as in DNA replication and repair. PCNA gene expression has been shown to be activated by plant specific transcription factors, PCFs. The regulatory elements of the gene confer cell division and/or proliferation specific expression that is also preferentially expressed in the immature ear and early kernel tissue of maize.

In accordance with the invention, nucleotide sequences are provided that allow initiation of transcription in actively dividing tissues. The sequences of the invention comprise transcriptional initiation regions associated with PCNA2 expression. Thus, the compositions of the present invention comprise novel nucleotide sequences for plant regulatory elements natively associated with the nucleotide sequences coding for maize PCNA2.

A method for expressing an isolated nucleotide sequence in a plant using the transcriptional initiation sequences disclosed herein is provided. The method comprises transforming a plant cell with a transformation vector that comprises an isolated nucleotide sequence operably linked to one or more of the plant regulatory sequences of the present invention and regenerating a stably transformed plant from the transformed plant cell. In this manner, the regulatory sequences are useful for controlling the expression of endogenous as well as exogenous products in a cell division and/or cell proliferation or even immature ear and early kernel tissue preferred manner.

Typically under the transcriptional initiation regulation of the elements of the invention will be a sequence of interest, which will provide for modification of the phenotype of the dividing cells. Such modification includes modulating the production of an endogenous product, as to amount, relative distribution, or the like, or production of an exogenous expression product to provide for a novel function or product in the actively dividing cells, or even suppression of endogenous products.

By “cell division, cell proliferation, or actively dividing cells” is intended any cells, tissue or organ in a plant which are actively involved in proliferation as evidenced by cells undergoing DNA replication, synthesis or repair.

By “immature ear and early kernel tissue” is intended any tissue of the female inflorescence indicating ovule and silk or the kernel including the tissues which will mature into the pericarp, aleurone, endosperm, scutellum, coleoptile, internode, endosperm at any time prior to maturity of the kernel.

By “regulatory element” is intended sequences responsible for tissue and temporal expression of the associated coding sequence including promoters, terminators, enhancers, introns, and the like.

By “terminator” is intended sequences that are needed for termination of transcription. A regulatory region of DNA that causes RNA polymerase to disassociate from DNA, causing termination of transcription.

By “promoter” is intended a regulatory element, or region of DNA usually comprising a TATA box capable of directing RNA polymerase II to initiate RNA synthesis at the appropriate transcription initiation site for a particular coding sequence. A promoter can additionally comprise other recognition sequences generally positioned upstream or 5′ to the TATA box, referred to as upstream promoter elements, which influence the transcription initiation rate. It is recognized that having identified the nucleotide sequences for the promoter region disclosed herein, it is within the state of the art to isolate and identify further regulatory elements in the 5′ untranslated region upstream from the particular promoter region identified herein. Thus the promoter region disclosed herein is generally further defined by comprising upstream regulatory elements such as those responsible for tissue and temporal expression of the coding sequence, enhancers and the like. In the same manner, the promoter elements which enable expression in the desired tissue comprising dividing cells can be identified, isolated, and used with other core promoters to confirm cellular division and/or cell proliferation-preferred expression.

The isolated regulatory elements (promoters sequences) of the present invention can be modified to provide for a range of expression levels of any isolated nucleotide sequence. Less than the entire promoter region can be utilized and the ability to drive cell division and or cell proliferation, or early ear/kernel preferred expression retained. However, it is recognized that expression levels of mRNA can be decreased with deletions of portions of the promoter sequence. Thus, the promoter can be modified to be a weak or strong promoter. Generally, by “weak promoter” is intended a promoter that drives expression of a coding sequence at a low level. By “low level” is intended levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts. Generally, at least about 20 nucleotides of an isolated promoter sequence will be used to drive expression of a nucleotide sequence.

It is recognized that to increase transcription levels enhancers can be utilized in combination with the promoter regions of the invention. Enhancers are nucleotide sequences that act to increase the expression of a promoter region. Enhancers are known in the art and include the SV40 enhancer region, the 35S enhancer element, and the like.

The regulatory elements of the present invention can be isolated from the 5′ untranslated region flanking its respective transcription initiation site of a PCNA gene. Likewise the terminator can be isolated from the 3′ untranslated region flanking its respective stop codon. The term “isolated” refers to material, such as a nucleic acid or protein, which is: (1) substantially or essentially free from components which normally accompany or interact with the material as found in its naturally occurring environment or (2) if the material is in its natural environment, the material has been altered by deliberate human intervention to a composition and/or placed at a locus in a cell other than the locus native to the material. Methods for isolation of promoter regions are well known in the art. One method is described in U.S. patent application Ser. No. 06/098,690 filed Aug. 31, 1998 herein incorporated by reference. The sequences for the promoter region is set forth in SEQ ID NO: 1.

The PCNA2 promoter set forth in SEQ ID NO: 1 is approximately 900 ID nucleotides in length (SEQ ID NO: 1). The regulatory element was isolated upstream from a PCNA2 coding sequence in maize.

A plasmids with the regulatory promoter PH018978 were also developed.

The promoter regions of the invention may be isolated from any plant, including, but not limited to corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), oats, barley, vegetables, ornamentals, and conifers. Preferably, plants include corn, soybean, sunflower, safflower, canola, wheat, barley, rye, alfalfa, and sorghum.

Promoter sequences from other plants may be isolated according to well-known techniques based on their sequence homology to the promoter sequences set forth herein. In these techniques, all or part of the known promoter sequence is used as a probe which selectively hybridizes to other sequences present in a population of cloned genomic DNA fragments (i.e. genomic libraries) from a chosen organism. Methods are readily available in the art for the hybridization of nucleic acid sequences.

The entire promoter sequence or portions thereof can be used as a probe capable of specifically hybridizing to corresponding promoter sequences. To achieve specific hybridization under a variety of conditions, such probes include sequences that are unique and are preferably at least about 10 nucleotides in length, and most preferably at least about 20 nucleotides in length. Such probes can be used to amplify corresponding promoter sequences from a chosen organism by the well-known process of polymerase chain reaction (PCR). This technique can be used to isolate additional promoter sequences from a desired organism or as a diagnostic assay to determine the presence of the promoter sequence in an organism. Examples include hybridization screening of plated DNA libraries (either plaques or colonies; see, e.g., Innis, et al., (1990) PCR Protocols, A Guide to Methods and Applications, eds., Academic Press).

The terms “stringent conditions” or “stringent hybridization conditions” includes reference to conditions under which a probe will hybridize to its target sequence, to a detectably greater degree than other sequences (e.g., at least 2-fold over background). Stringent conditions are target-sequence dependent and will differ depending on the structure of the polynucleotide. By controlling the stringency of the hybridization and/or washing conditions, target sequences can be identified which are 100% complementary to a probe (homologous probing). Alternatively, stringency conditions can be adjusted to allow some mismatching in sequences so that lower degrees of similarity are detected (heterologous probing). Generally, probes of this type are in a range of about 250 nucleotides in length to about 1000 nucleotides in length.

An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995). See also, Sambrook, et al., (1989) Molecular Cloning: A Laboratory Manual (2nd ed. Cold Spring Harbor Laboratory, Cold Spring Harbor, N.Y.).

In general, sequences that correspond to the promoter sequence of the present invention and hybridize to the promoter sequence disclosed herein will be at least 50% homologous, 55% homologous, 60% homologous, 65% homologous, 70% homologous, 75% homologous, 80% homologous, 85% homologous, 90% homologous, 95% homologous and even 98% homologous or more with the disclosed sequence.

Specificity is typically the function of post-hybridization washes, the critical factors being the ionic strength and temperature of the final wash solution. Generally, stringent wash temperature conditions are selected to be about 5° C. to about 2° C. lower than the melting point (Tₘ) for the specific sequence at a defined ionic strength and pH. The melting point, or denaturation, of DNA occurs over a narrow temperature range and represents the disruption of the double helix into its complementary single strands. The process is described by the temperature of the midpoint of transition, Tₘ, which is also called the melting temperature. Formulas are available in the art for the determination of melting temperatures.

Hybridization conditions for the promoter sequences of the invention include hybridization at 42° C. in 50% (w/v) formamide, 6×SSC, 0.5% (w/v) SDS, 100 μg/ml salmon sperm DNA. Exemplary low stringency washing conditions include hybridization at 42° C. in a solution of 2×SSC, 0.5% (w/v) SDS for 30 minutes and repeating. Exemplary moderate stringency conditions include a wash in 2×SSC, 0.5% (w/v) SDS at 50° C. for 30 minutes and repeating. Exemplary high stringency conditions include a wash in 2×SSC, 0.5% (w/v) SDS, at 65° C. for 30 minutes and repeating. Sequences that correspond to the promoter of the present invention may be obtained using all the above conditions.

The following terms are used to describe the sequence relationships between two or more nucleic acids or polynucleotides: (a) “reference sequence”, (b) “comparison window”, (c) “percentage of sequence identity”, and (d) “substantial identity”.

(a) As used herein, “reference sequence” is a defined sequence used as a basis for sequence comparison. A reference sequence may be a subset or the entirety of a specified sequence; for example, as a segment of a full-length promoter sequence, or the complete promoter sequence.

(b) As used herein, “comparison window” makes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length and optionally can be 30, 40, 50, 100 or more contiguous nucleotides in length. Those of skill in the art understand that to avoid a high similarity to a reference sequence due to inclusion of gaps in the polynucleotide sequence a gap penalty is typically introduced and is subtracted from the number of matches.

(c) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

(d) The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters.

Methods of aligning sequences for comparison are well known in the art. Gene comparisons can be determined by conducting BLAST (Basic Local Alignment Search Tool; Altschul, et al., (1993) J. Mol. Biol. 215:403-410; see also, www.ncbi.nlm.nih.gov/BLAST/) searches under default parameters for identity to sequences contained in the BLAST “GENEMBL” database. A sequence can be analyzed for identity to all publicly available DNA sequences contained in the GENEMBL database using the BLASTN algorithm under the default parameters. Identity to the sequence of the present invention would mean a polynucleotide sequence having at least 65% sequence identity, more preferably at least 70% sequence identity, more preferably at least 75% sequence identity, more preferably at least 80% identity, more preferably at least 85% sequence identity, more preferably at least 90% sequence identity and most preferably at least 95% sequence identity wherein the percent sequence identity is based on the entire promoter region.

GAP uses the algorithm of Needleman and Wunsch, (J. Mol. Biol. 48:443-453, 1970) to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

Sequence fragments with high percent identity to the sequences of the present invention also refer to those fragments of a particular regulatory element nucleotide sequence disclosed herein that operate to promote the cell division-preferred expression of an operably linked isolated nucleotide sequence. These fragments will comprise at least about 20 contiguous nucleotides, preferably at least about 50 contiguous nucleotides, more preferably at least about 75 contiguous nucleotides, even more preferably at least about 100 contiguous nucleotides of the particular promoter nucleotide sequence disclosed herein. The nucleotides of such fragments will usually comprise the TATA recognition sequence of the particular promoter sequence. Such fragments can be obtained by use of restriction enzymes to cleave the naturally occurring regulatory element nucleotide sequences disclosed herein; by synthesizing a nucleotide sequence from the naturally occurring DNA sequence; or can be obtained through the use of PCR technology. See particularly, Mullis, et al., (1987) Methods Enzymol. 155:335-350, and Erlich, ed. (1989) PCR Technology (Stockton Press, New York). Again, variants of these fragments, such as those resulting from site-directed mutagenesis, are encompassed by the compositions of the present invention.

Nucleotide sequences comprising at least about 20 contiguous sequences of the sequence set forth in SEQ ID NOS: 1 or 2 are encompassed. These sequences can be isolated by hybridization, PCR, and the like. Such sequences encompass fragments capable of driving cell proliferation-preferred expression, fragments useful as probes to identify similar sequences, as well as elements responsible for temporal or tissue specificity.

Biologically active variants of the regulatory sequences are also encompassed by the compositions of the present invention. A regulatory “variant” is a modified form of a regulatory sequence wherein one or more bases have been modified, removed or added. For example, a routine way to remove part of a DNA sequence is to use an exonuclease in combination with DNA amplification to produce unidirectional nested deletions of double stranded DNA clones. A commercial kit for this purpose is sold under the trade name Exo-Size™ (New England Biolabs, Beverly, Mass.). Briefly, this procedure entails incubating exonuclease III with DNA to progressively remove nucleotides in the 3′ to 5′ direction at 5′ overhangs, blunt ends or nicks in the DNA template. However, exonuclease III is unable to remove nucleotides at 3′, 4-base overhangs. Timed digests of a clone with this enzyme produces unidirectional nested deletions.

One example of a regulatory sequence variant is a promoter formed by one or more deletions from a larger promoter. The 5′ portion of a promoter up to the TATA box near the transcription start site can be deleted without abolishing promoter activity, as described by Zhu, et al., (1995) The Plant Cell 7:1681-89. Such variants should retain promoter activity, particularly the ability to drive expression in actively dividing cells and their tissues. Biologically active variants include, for example, the native regulatory sequences of the invention having one or more nucleotide substitutions, deletions or insertions. Activity can be measured by Northern blot analysis, reporter activity measurements when using transcriptional fusions, and the like. See, for example, Sambrook, et al., (1989) Molecular Cloning: A Laboratory Manual (2nd ed. Cold Spring Harbor Laboratory, Cold Spring Harbor, N.Y.), herein incorporated by reference.

The nucleotide sequences for the cell division or cell proliferation-preferred regulatory elements disclosed in the present invention, as well as variants and fragments thereof, are useful in the genetic manipulation of any plant when operably linked with an isolated nucleotide sequence whose expression is to be controlled to achieve a desired phenotypic response. By “operably linked” is intended the transcription or translation of the isolated nucleotide sequence is under the influence of the regulatory sequence. In this manner, the nucleotide sequences for the regulatory elements of the invention may be provided in expression cassettes along with isolated nucleotide sequences for expression in the plant of interest, more particularly in the actively dividing cells of the plant. Such an expression cassette is provided with a plurality of restriction sites for insertion of the nucleotide sequence to be under the transcriptional control of the regulatory elements.

The genes of interest expressed by the regulatory elements of the invention can be used for varying the phenotype of tissues as they undergo periods of cell division or proliferation. This can be achieved by increasing expression of endogenous or exogenous products in these tissues. Alternatively, the results can be achieved by providing for a reduction of expression of one or more endogenous products, particularly enzymes or cofactors in the dividing or proliferating tissue. These modifications result in a change in phenotype of the transformed tissue or plant. It is recognized that the regulatory elements may be used with their native coding sequences to increase or decrease expression resulting in a change in phenotype in the transformed plant or tissue.

In another embodiment, the regulatory elements of the invention can be used for proliferating cell-preferred expression of selectable markers. For example, regulatory elements such as the Led promoter and terminator would allow plants to be regenerated that have no field resistance to herbicide but may be completely susceptible to the herbicide in the actively dividing stage.

General categories of genes of interest for the purposes of the present invention include for example, those genes involved in information, such as Zinc fingers; those involved in communication, such as kinases; and those involved in housekeeping, such as heat shock proteins. More specific categories of transgenes include genes encoding important traits for agronomics, insect resistance, disease resistance, herbicide resistance, and grain characteristics. Still other categories of transgenes include genes for inducing expression of exogenous products such as enzymes, cofactors, and hormones from plants and other eukaryotes as well as prokaryotic organisms. It is recognized that any gene of interest, including the native coding sequence, can be operably linked to the regulatory elements of the invention and expressed in the plant.

Modifications that affect grain traits include increasing the content of oleic acid, or altering levels of saturated and unsaturated fatty acids. Likewise, increasing the levels of lysine and sulfur-containing amino acids may be desired as well as the modification of starch type and content in the seed. Hordothionin protein modifications are described in WO 9416078 filed Apr. 10, 1997; WO 9638562 filed Mar. 26, 1997; WO 9638563 filed Mar. 26, 1997 and U.S. Pat. No. 5,703,409 issued Dec. 30, 1997; the disclosures of which are incorporated herein by reference. Another example is lysine and/or sulfur-rich seed protein encoded by the soybean 2S albumin described in WO 9735023 filed Mar. 20, 1996, and the chymotrypsin inhibitor from barley, Williamson, et al., (1987) Eur. J. Biochem. 165:99-106, the disclosures of each are incorporated by reference.

Derivatives of the following genes can be made by site-directed mutagenesis to increase the level of preselected amino acids in the encoded polypeptide. For example, the gene encoding the barley high lysine polypeptide (BHL) is derived from barley chymotrypsin inhibitor, WO 9820133 filed Nov. 1, 1996 the disclosure of which is incorporated herein by reference. Other proteins include methionine-rich plant proteins such as from sunflower seed, Lilley, et al., (1989) Proceedings of the World Congress on Vegetable Protein Utilization in Human Foods and Animal Feedstuffs; Applewhite, (ed.); American Oil Chemists Soc., Champaign, Ill.:497-502, incorporated herein by reference; corn, Pedersen, et al., (1986) J. Biol. Chem. 261:6279; Kirihara, et al., (1988) Gene 71:359, both incorporated herein by reference; and rice, Musumura, et al., (1989) Plant Mol. Biol. 12:123, incorporated herein by reference. Other important genes encode glucans, Floury 2, growth factors, seed storage factors and transcription factors.

Agronomic traits in plants can be improved by altering expression of genes that: affect the response of plant growth and development during environmental stress, Cheikh-N, et al., (1994) Plant Physiol. 106(1):45-51) and genes controlling carbohydrate metabolism to reduce kernel abortion in maize, Zinselmeier, et al., (1995) Plant Physiol. 107(2):385-391.

Insect resistance genes may encode resistance to pests that have great yield drag such as rootworm, cutworm, European Corn Borer, and the like. Such genes include, for example: Bacillus thuringiensis endotoxin genes, U.S. Pat. Nos. 5,366,892; 5,747,450; 5,737,514; 5,723,756; 5,593,881; Geiser, et al., (1986) Gene 48:109; lectins, Van Damme, et al., (1994) Plant Mol. Biol. 24:825; and the like.

Genes encoding disease resistance traits include: detoxification genes, such as against fumonosin (WO 9606175 filed Jun. 7, 1995); avirulence (avr) and disease resistance (R) genes, Jones, et al., (1994) Science 266:789; Martin, et al., (1993) Science 262:1432; Mindrinos, et al., (1994) Cell 78:1089; and the like.

Commercial traits can also be encoded on a gene(s) which could alter or increase for example, starch for the production of paper, textiles and ethanol, or provide expression of proteins with other commercial uses. Another important commercial use of transformed plants is the production of polymers and bioplastics such as described in U.S. Pat. No. 5,602,321 issued Feb. 11, 1997. Genes such as B-Ketothiolase, PHBase (polyhydroxyburyrate synthase) and acetoacetyl-CoA reductase (see, Schubert, et al., (1988) J. Bacteriol 170(12):5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like.

The nucleotide sequence operably linked to the regulatory elements disclosed herein can be an antisense sequence for a targeted gene. By “antisense DNA nucleotide sequence” is intended a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing with the endogenous messenger RNA (mRNA) produced by transcription of the DNA nucleotide sequence for the targeted gene. In this case, production of the native protein encoded by the targeted gene is inhibited to achieve a desired phenotypic response. Thus the regulatory sequences disclosed herein can be operably linked to antisense DNA sequences to reduce or inhibit expression of a native protein in the plant.

The expression cassette will also include at the 3′ terminus of the isolated nucleotide sequence of interest, a transcriptional and translational termination region functional in plants. The termination region can be native with the promoter nucleotide sequence of the present invention, can be native with the DNA sequence of interest, or can be derived from another source.

Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., 1989) Nucleic Acids Res. 17:7891-7903; Joshi, et al., (1987) Nucleic Acid Res. 15:9627-9639.

The expression cassettes can additionally contain 5′ leader sequences. Such leader sequences can act to enhance translation. Translation leaders are known in the art and include: picornavirus leaders, for example: EMCV leader (Encephalomyocarditis 5′ noncoding region), Elroy-Stein, et al., (1989) Proc. Nat. Acad. Sci. USA 86:6126-6130; potyvirus leaders, for example, TEV leader (Tobacco Etch Virus), Allison, et al., (1986); MDMV leader (Maize Dwarf Mosaic Virus), Virology 154:9-20; human immunoglobulin heavy-chain binding protein (BiP), Macejak, et al., (1991) Nature 353:90-94; untranslated leader from the coat protein mRNA of alfalfa mosaic virus (AMV RNA 4), Jobling, et al., (1987) Nature 325:622-625); tobacco mosaic virus leader (TMV), Gallie, et al., (1989) Molecular Biology of RNA, pages 237-256; and maize chlorotic mottle virus leader (MCMV) Lommel, et al., (1991) Virology 81:382-385. See also, Della-Cioppa, et al., (1987) Plant Physiology 84:965-968. The cassette can also contain sequences that enhance translation and/or mRNA stability such as introns.

In those instances where it is desirable to have the expressed product of the isolated nucleotide sequence directed to a particular organelle, particularly the plastid, amyloplast, or to the endoplasmic reticulum, or secreted at the cell's surface or extracellularly, the expression cassette can further comprise a coding sequence for a transit peptide. Such transit peptides are well known in the art and include, but are not limited to: the transit peptide for the acyl carrier protein, the small subunit of RUBISCO, plant EPSP synthase, and the like.

In preparing the expression cassette, the various DNA fragments can be manipulated, so as to provide for the DNA sequences in the proper orientation and, as appropriate, in the proper reading frame. Toward this end, adapters or linkers can be employed to join the DNA fragments or other manipulations can be involved to provide for convenient restriction sites, removal of superfluous DNA, removal of restriction sites, or the like. For this purpose, in vitro mutagenesis, primer repair, restriction digests, annealing, and resubstitutions such as transitions and transversions, can be involved.

As noted herein, the present invention provides vectors capable of expressing genes of interest under the control of the regulatory elements. In general, the vectors should be functional in plant cells. At times, it may be preferable to have vectors that are functional in E. coli (e.g., production of protein for raising antibodies, DNA sequence analysis, construction of inserts, obtaining quantities of nucleic acids). Vectors and procedures for cloning and expression in E. coli are discussed in Sambrook, et al. (supra).

The transformation vector comprising the regulatory sequences of the present invention operably linked to an isolated nucleotide sequence in an expression cassette, can also contain at least one additional nucleotide sequence for a gene to be cotransformed into the organism. Alternatively, the additional sequence(s) can be provided on another transformation vector.

Vectors that are functional in plants can be binary plasmids derived from Agrobacterium. Such vectors are capable of transforming plant cells. These vectors contain left and right border sequences that are required for integration into the host (plant) chromosome. At minimum, between these border sequences is the gene to be expressed under control of the regulatory elements of the present invention. In one embodiment, a selectable marker and a reporter gene are also included. For ease of obtaining sufficient quantities of vector, a bacterial origin that allows replication in E. coli can be used.

Reporter genes can be included in the transformation vectors. Examples of suitable reporter genes known in the art can be found in, for example: Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) BioTechniques 19:650-655; and Chiu, et al., (1996) Current Biology 6:325-330.

Selectable marker genes for selection of transformed cells or tissues can be included in the transformation vectors. These can include genes that confer antibiotic resistance or resistance to herbicides. Examples of suitable selectable marker genes include, but are not limited to: genes encoding resistance to chloramphenicol, Herrera Estrella, et al., (1983) EMBO J. 2:987-992; methotrexate, Herrera Estrella, et al., (1983) Nature 303:209-213; Meijer, et al., (1991) Plant Mol. Biol. 16:807-820; hygromycin, Waldron, et al., (1985) Plant Mol. Biol. 5:103-108; Zhijian, et al., (1995) Plant Science 108:219-227; streptomycin, Jones, et al., (1987) Mol. Gen. Genet. 210:86-91; spectinomycin, Bretagne-Sagnard, et al., (1996) Transgenic Res. 5:131-137; bleomycin, Hille, et al., (1990) Plant Mol. Biol. 7:171-176; sulfonamide, Guerineau, et al., (1990) Plant Mol. Biol. 15:127-136; bromoxynil, Stalker, et al., (1988) Science 242:419-423; glyphosate, Shaw, et al., (1986) Science 233:478-481; phosphinothricin, DeBlock, et al., (1987) EMBO J. 6:2513-2518.

Other genes that could serve utility in the recovery of transgenic events but might not be required in the final product would include, but are not limited to: GUS (β-glucoronidase), Jefferson, (1987) Plant Mol. Biol. Rep. 5:387); GFP (green florescence protein), Chalfie, et al., (1994) Science 263:802; luciferase, Teeri, et al., (1989) EMBO J. 8:343; and the maize genes encoding for anthocyanin production, Ludwig, et al., (1990) Science 247:449.

The transformation vector comprising the particular regulatory sequences of the present invention, operably linked to an isolated nucleotide sequence of interest in an expression cassette, can be used to transform any plant. In this manner, genetically modified plants, plant cells, plant tissue, seed, and the like can be obtained. Transformation protocols can vary depending on the type of plant or plant cell, i.e., monocot or dicot, targeted for transformation. Suitable methods of transforming plant cells include microinjection, Crossway, et al., (1986) Biotechniques 4:320-334; electroporation, Riggs, et al., (1986) Proc. Natl. Acad. Sci. USA 83:5602-5606; Agrobacterium-mediated transformation, see for example, Townsend, et al., U.S. Pat. No. 5,563,055; direct gene transfer, Paszkowski, et al., (1984) EMBO J. 3:2717-2722; and ballistic particle acceleration, see for example, Sanford, et al., U.S. Pat. No. 4,945,050; Tomes, et al., (1995) in Plant Cell, Tissue, and Organ Culture: Fundamental Methods, ed. Gamborg and Phillips (Springer-Verlag, Berlin); and McCabe, et al., (1988) Biotechnology 6:923-926. Also see, Weissinger, et al., (1988) Annual Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); Klein, et al., (1988) Plant Physiol. 91:440-444 (maize); Fromm, et al., (1990) Biotechnology 8:833-839; Hooydaas-Van Slogteren, et al., (1984) Nature (London) 311:763-764; Bytebier, et al., (1987) Proc. Natl. Acad. Sci. USA 84:5345-5349 (Liliaceae); De Wet, et al., (1985) in The Experimental Manipulation of Ovule Tissues, ed. G. P. Chapman, et al., (Longman, New York), pp. 197-209 (pollen); Kaeppler, et al., (1990) Plant Cell Reports 9:415-418; and Kaeppler, et al., (1992) Theor. Appl. Genet. 84:560-566 (whisker-mediated transformation); D. Halluin, et al., (1992) Plant Cell 4:1495-1505 (electroporation); Li, et al., (1993) Plant Cell Reports 12:250-255 and Christou, et al., (1995) Annals of Botany 75:407-413 (rice); Osjoda, et al., (1996) Nature Biotechnology 14:745-750 (maize via Agrobacterium tumefaciens); all of which are herein incorporated by reference.

The cells that have been transformed can be grown into plants in accordance with conventional ways. See, for example, McCormick, et al., (1986) Plant Cell Reports 5:81-84. These plants can then be grown and pollinated with the same transformed strain or different strains. The resulting hybrid having cellular division and/or proliferation-preferred expression of the desired phenotypic characteristic can then be identified. Two or more generations can be grown to ensure that cell division or proliferation-preferred expression of the desired phenotypic characteristic is stably maintained and inherited.

The following examples are offered by way of illustration and not by way of limitation.

### EXAMPLES

### Example 1

### Genomic DNA Isolation

The genomic sequence including the regulatory elements of the invention were isolated using methods described in the User Manual for the Genome Walker kit sold by Clontech Laboratories, Inc., Palo Alto, Calif. Genomic DNA upstream of the coding sequence for the maize PCNA2 gene was isolated using this method.

### Northern Blot

RNA was isolated from shoots of 4-week seedlings using the TriZol method (Invitrogen, Carlsbad, Calif.). 15 ug total RNA was separated on 1% agrarose MOPS-formaldehyde gels and blotted on Hybond-N+ membrane (Amersham). DNA probes were labeled using RediPrimeII kit (Amersham) and hybridized to membrane in ExpressHyb (CLONTECH, Palo Alto, Calif.) at 65° C. overnight. The membranes were washed twice in 2×SSC, 0.1% SDS at room temperature and twice in 0.1×SSC, 0.1% SDS at 50° C. The membranes were autographed to visualize hybridization signals.

### Results

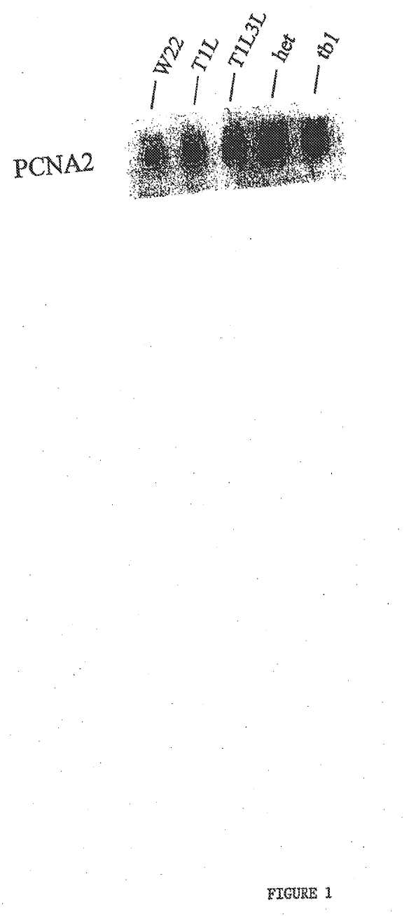

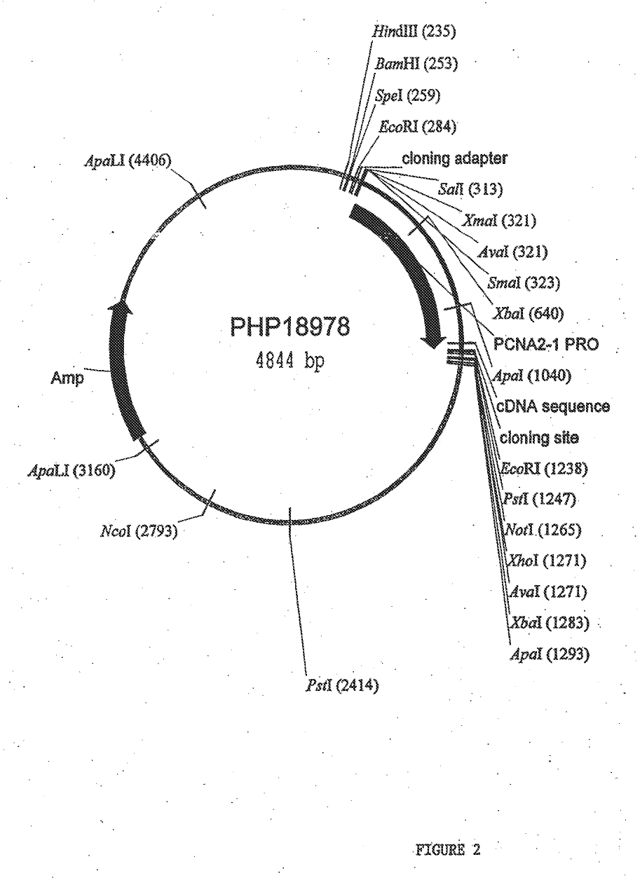

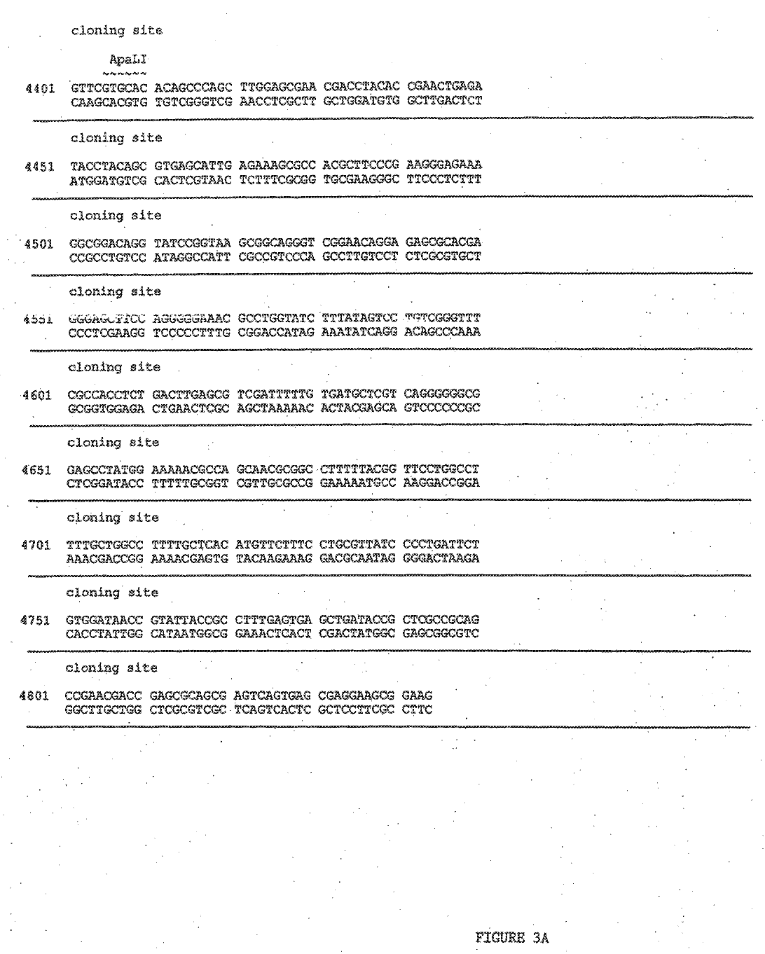

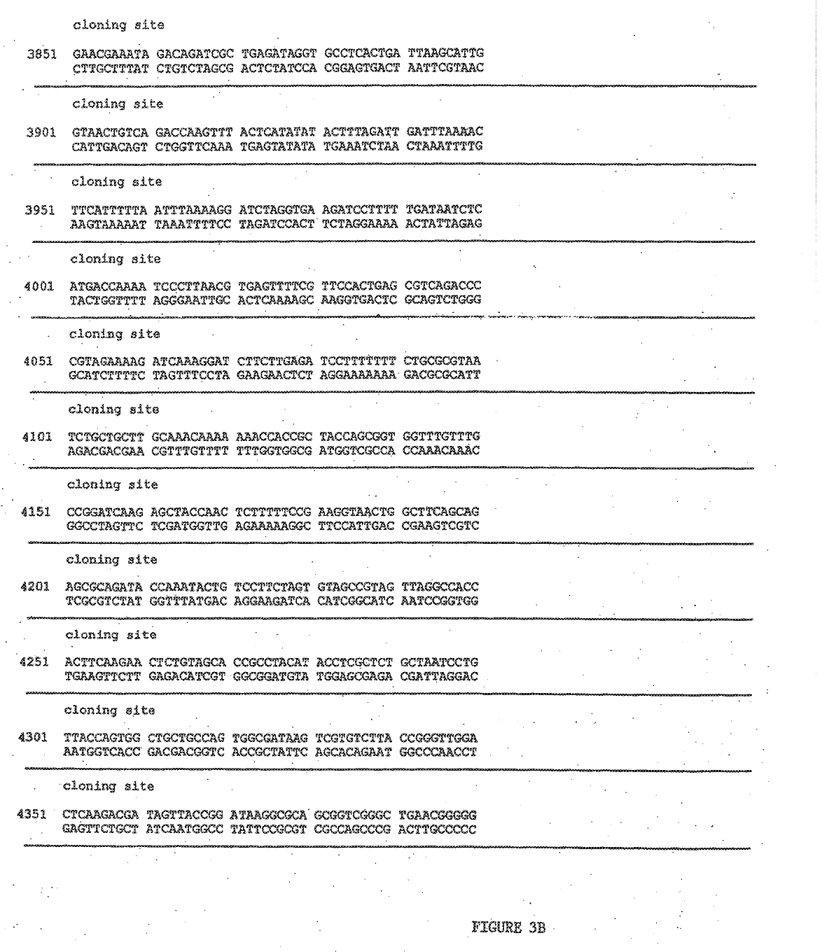

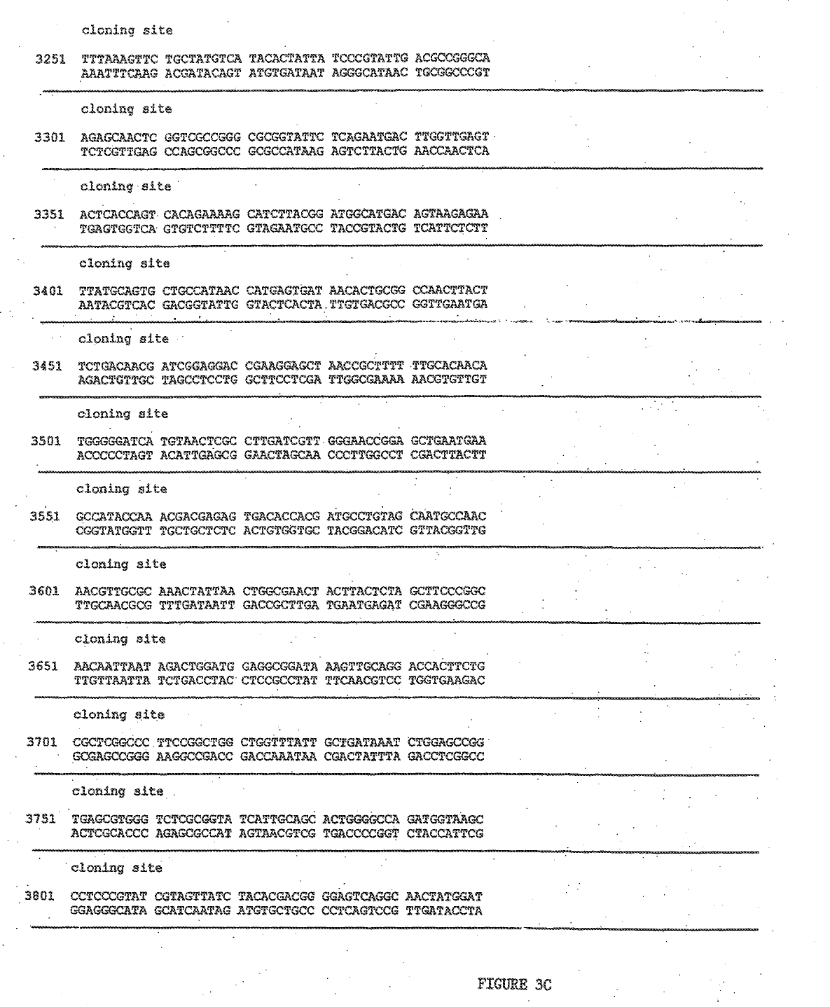

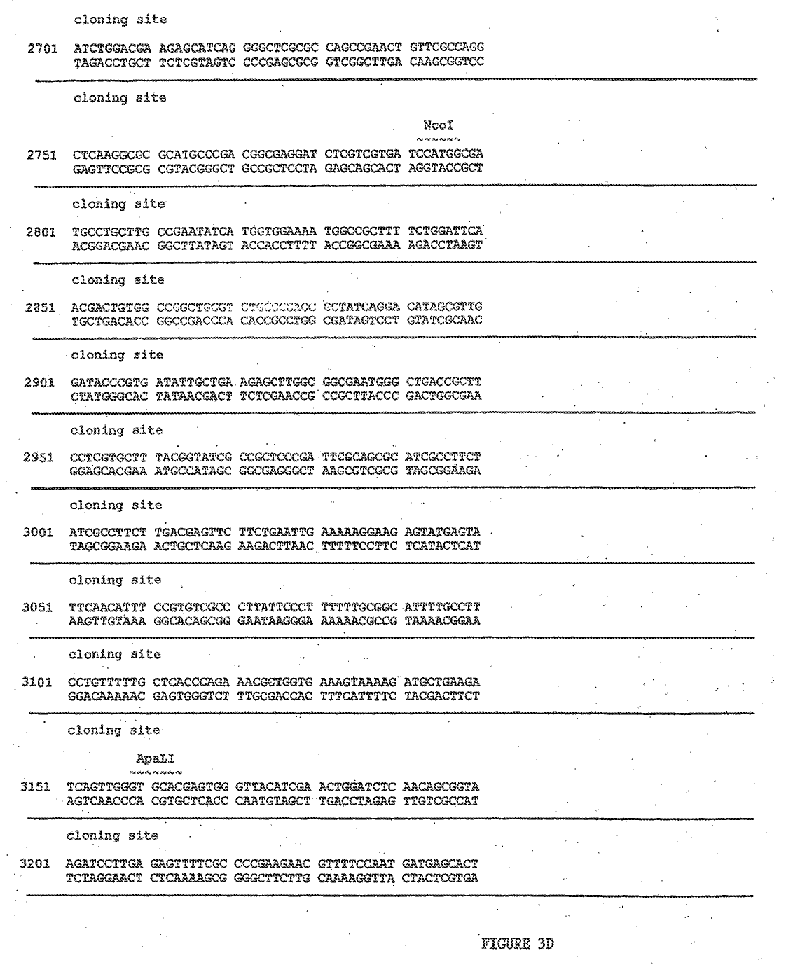

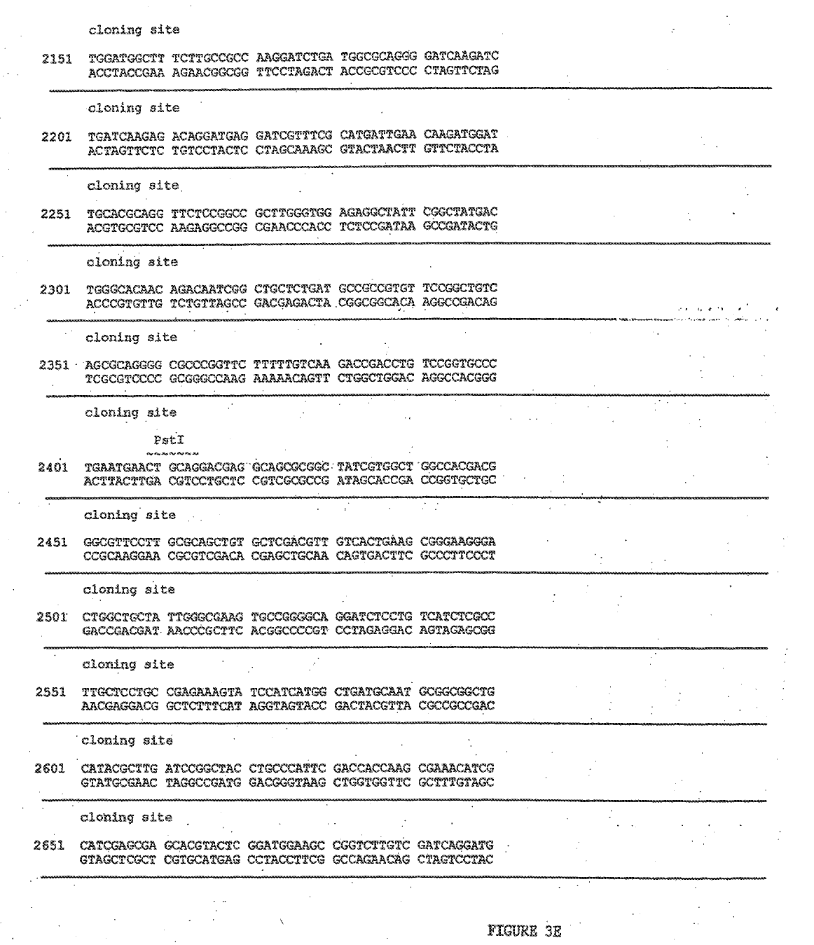

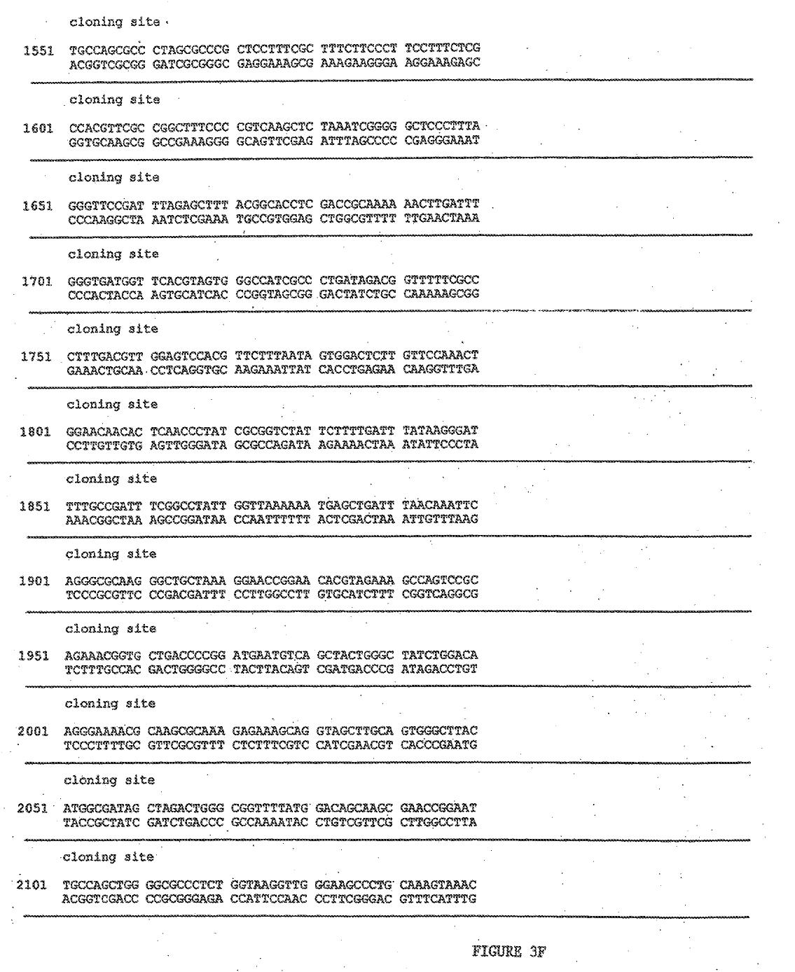

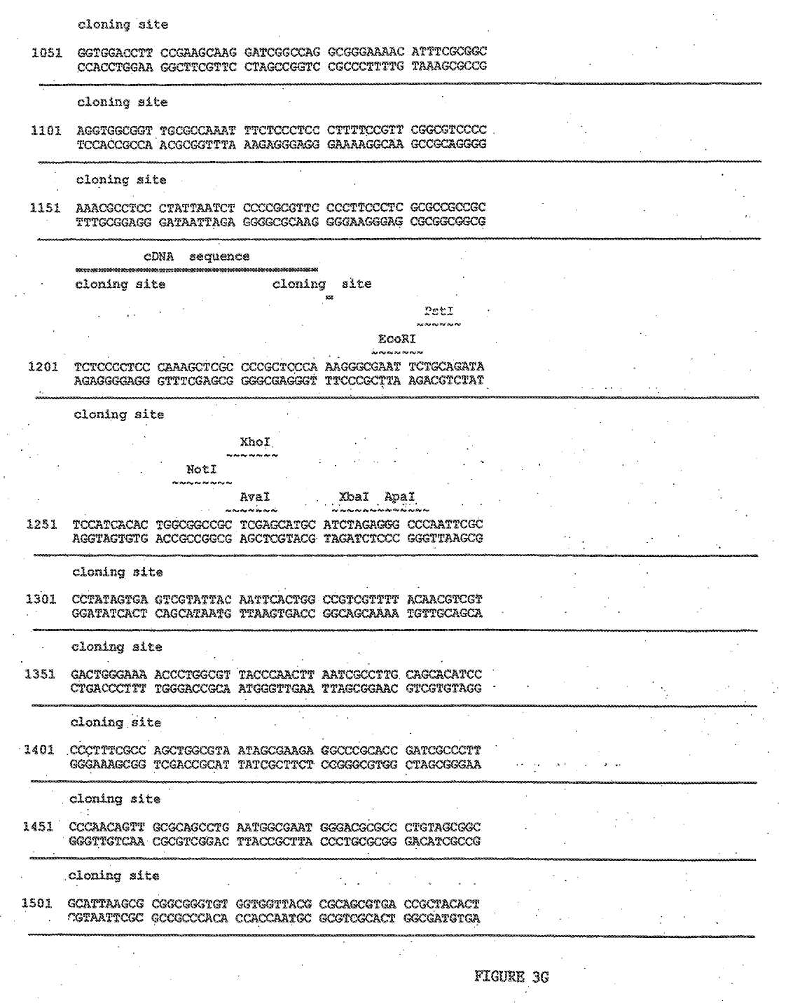

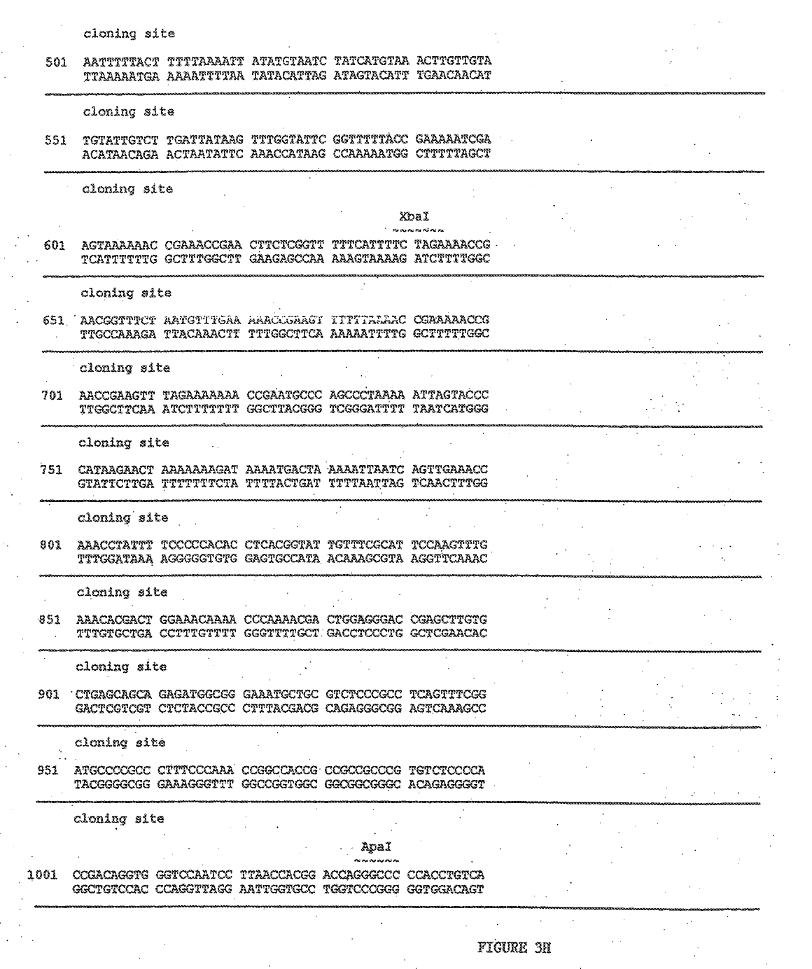

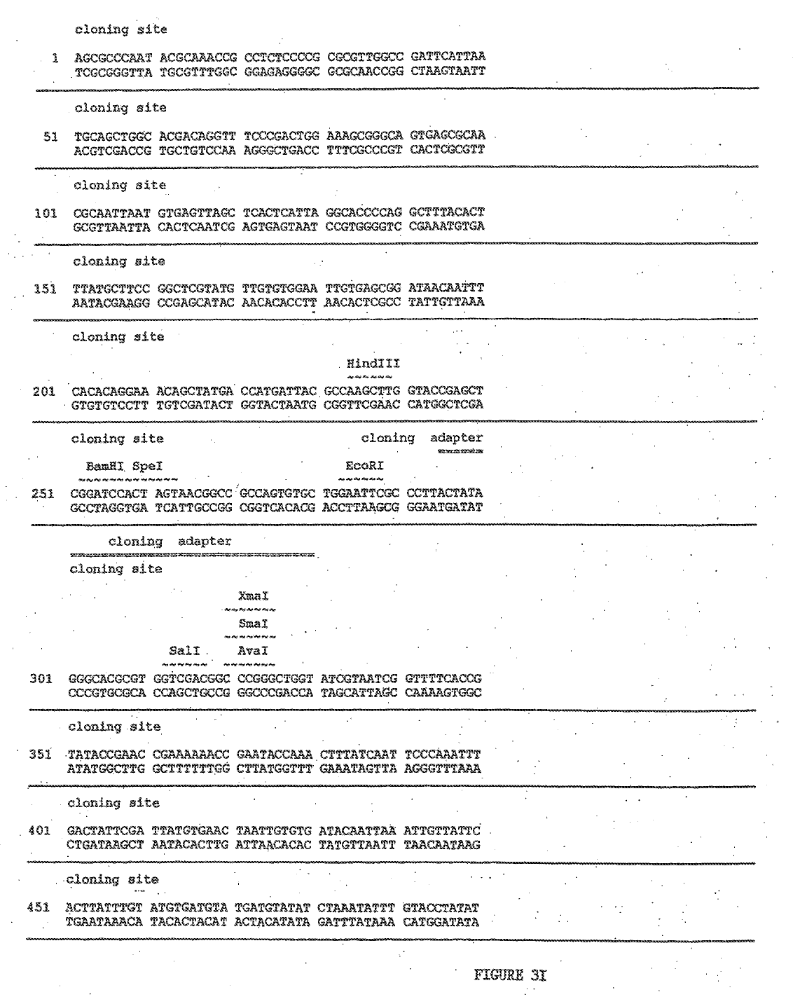

See FIG. 1 for the northern analysis of PCN2 gene expression in wild type plant (W22), W22 plant introgressed with the teosinate 1L chromosome (T1L), W22 plant introgressed with the teosinate 1L and 3L chromosomes (T1L3L), Tb1/tb1-mum3 heterozygote (het) and tb1-mum3 homozygote (tb1). All are in W22 background.

### Example 2

### PCNA2 Expression

| PPM | |
|---|---|
| Adj | Title |
| 867 | B73, immature ear (5-10 mm), base |
| 806 | ear tip, immature ear |
| 799 | EE3DT, immature ear, V11 |
| 604 | EE09B, immature ear, V11 |
| 595 | Mo17, immature ear |
| 536 | Corn HG11 10 DAP tissue cultured embryos, non responsive |
| 523 | EE3DT, immature ear, V12 |
| 483 | Corn embryos B73, 15 DAP |
| 441 | B73/Mo17, immature ear |
| 438 | B73, immature ear |
| 428 | Corn immature ear at R1/silking |
| 425 | Corn HG11 10 DAP tissue cultured embryos, responsive |
| 424 | Mo17/B73, immature ear |
| 393 | Corn embryos Qx47, 15 DAP |
| 347 | Corn embryos Illinois High Oil, 15 DAP |
| 322 | Corn immature ear at R1/silking |
| 320 | ear base, immature ear |
| 305 | B73 endosperm, 6 DAP embryo sac |
| 303 | Corn immature 1o and 2o ear shoots, V11 |
| 300 | Corn embryos Askc0, 15 DAP |
| 290 | Corn 2 cm tassel + 4 cm tassel, V8-V10 |
| 251 | EE09B, immature ear, V12 |
| 238 | Corn embryos Askc28, 15 DAP |
| 238 | embryo axis, 20DAP |
| 201 | Corn endosperm 8 DAP |
| 176 | Corn B73 stalk |
| 170 | Corn ears 6-8 hrs. after pollination, V15 |
| 151 | Corn 6-day roots with 1-day 20 uM ABA induction |
| 144 | Corn 6-day roots without ABA induction |
| 137 | Corn whole kernels, embryo and endosperm, 0DAP |
| 129 | B73, 28K, nodal plate + pulvinus + rind/elongation zone |
| 119 | Corn embryo 21 DAP |
| 106 | 30 DAP embryo, B73 |
| 105 | Corn embryos, AGP transgenics |
| 99 | Corn embryos, AGP wild type |
| 85 | Corn tassel, meiosis I/II |
| 75 | Corn 10-day roots grown on 7% sucrose |
| 72 | Corn endosperm 12 DAP |
| 71 | Corn primary root, V2 |
| 70 | Corn 10-day roots grown on 2% sucrose |
| 68 | Corn root, test experiment |
| 59 | B73 scutellum |
| 56 | Corn pedicels control |
| 50 | Corn whole kernels, embryo and endosperm, 8DAP |
| 50 | Corn developing tassel wild type |
| 45 | Corn soft endosperm, 20-25-30 DAP |
| 45 | Corn silk, preemergent stage |
| 42 | Corn stem, sheath, V7-8 |
| 42 | Corn pericarp, white, 22DAP, Co63P1-ww |
| 37 | Corn developing tassel male sterile mutant ms22 |
| 35 | Corn embryo 35 DAP |
| 34 | pericarp, early, 15DAP B73 |
| 30 | Corn Adventitious/whole roots, V12-R1 |
| 27 | Corn primary root, V2 |
| 24 | pericarp, mid, 27DAP, B73 |
| 24 | Corn soft endosperm, B73 |
| 23 | Corn pedicels drought-stressed |
| 23 | Corn silk, 2 h post-pollination |
| 19 | Corn endosperm 21 DAP |
| 16 | Corn endosperm 35 DAP |
| 14 | 40 DAP embryo |
| 12 | Corn seedling mature mesocotyl, 5 days |
| 11 | Corn endosperm and pericarp, early develop. |
| | (30DAP = 8-10DAP) |
| 9 | Corn hard endosperm, 20-25-30 DAP |
| 8 | Corn pericarp, red, 22DAP, Co63P1-rr |
| 7 | Corn seedling, B73 X Mo17 F2-15 |

### Example 3

### Maize PCNA Gene Expression is Regulated by both PCF and Tb1 Transcription Factors

PCNA gene expression has been shown to be activated by plant specific bHLH transcription factors PCFs. Tb1 (Teosinate branched) has also been suggested to be in the same family (TCP family) as PCFs. Tb1 mutant plants display a dramatic phenotype similar to the maize progenitor teosinte, with extensively branched tillers as well as flowering effects. Here we demonstrate, by binding site selection and DNA binding studies, that Tb1 can also bind to the PCNA promoter at the same sites as for PCFs. Consistent with the notion that Tb1 functions as a repressor to inhibit maize lateral branch growth, tb1 mutant plants have an elevated level of PCNA gene expression. Since Tb1 does not heterodimerize with PCFs, Tb1 and PCFs compete for the same sites in the PCNA promoter to regulate PCNA gene expression.

Tb1 can bind to PCNA promoter DNA as determined by binding site selection and DNA binding studies.

PCNA gene expression is increased in tb1 mutant plants.

Tb1 does not heterodimerize with PCF transcription factors.

Mapping the PCNA genes does not correspond to a quantitative trait loci associated with domestication.

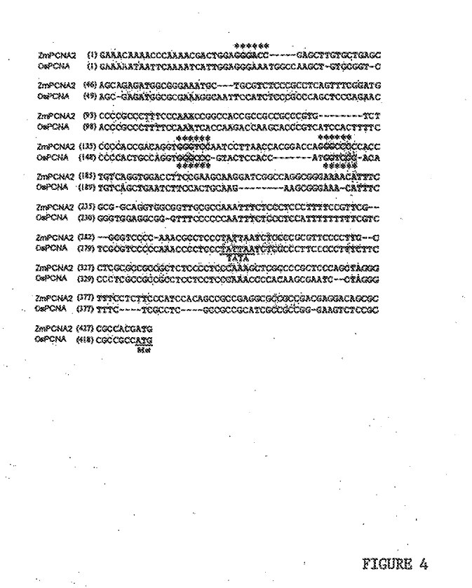

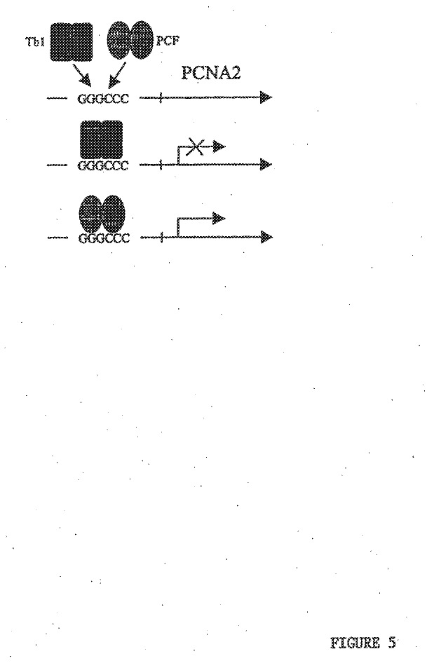

Thus the inventors conclude that Tb1 and PCNF compete for the same binding sites in the PCNA2 gene promoter. Under normal conditions, Tb1 occupies the binding sites; PCNA2 expression is blocked or reduced. When PCFs occupy the binding sites, PCHA2 gene expression is activated. See FIG. 4.

### REFERENCES

Doebley, J A, Stec, et. al., (1997) “The evolution of apical dominance in maize.” Nature 386(6624):485-8.
Cubas, P, N Lauter, et al., (1999) “The TCP domain: a motif found in proteins regulating plant growth and development.” Plant J. 18(2):215-22.
Kosugi, S and Y Ohashi, (1997). “PCF1 and PCF2 specifically bind to cis elements in the rice proliferating cell nuclear antigen gene.” Plant Cell 9(9):1607-19.

What is claimed is:

1. An isolated regulatory element that is capable of driving transcription in a cell division-preferred and/or proliferation-preferred manner, wherein said regulatory element comprises a nucleotide sequence selected from the group consisting of:
a) native sequences operably linked to DNA coding for maize PCNA2 (Proliferating Cell Nuclear Antigen 2);
b) the nucleotide sequence set forth in SEQ ID NO: 1;
c) the nucleotide sequence set forth in SEQ ID NO: 2, positions 861 through 1276; and
d) the nucleotide sequence set forth in SEQ ID NO: 2, positions 331 through 1230.

2. The isolated regulatory element of claim 1, wherein said regulatory element is capable of driving transcription in the immature ear and early kernel tissues of maize.

3. The isolated regulatory element of claim 2, wherein said regulatory element comprises one or more Tb1/PCF (teosinte branched 1/proliferating cell factor) binding sites.

4. The isolated regulatory element of claim 1 wherein said regulatory element comprises a nucleotide sequence which comprises a TATA box motif.

5. The isolated regulatory element of claim 1, wherein said regulatory element requires PCF binding for initiation of transcription.

6. An expression cassette comprising a nucleotide sequence operably linked to a regulatory element of claim 1.

7. A plasmid comprising the expression cassette of claim 6.

8. The plasmid of claim 7 wherein said plasmid is PHP18978.

9. A transformation vector comprising the expression cassette of claim 6.

10. A plant stably transformed with the expression cassette of claim 6.

11. The plant of claim 10, wherein said plant is a monocot.

12. The plant of claim 11, wherein said monocot is maize, wheat, rice, barley, sorghum, or rye.

13. A seed of the plant of claim 12, wherein the genome of said seed comprises at least a single copy of said expression cassette.

14. A method for selectively expressing a nucleotide sequence in a plant tissue which comprises actively dividing cells, the method comprising transforming a plant cell with a transformation vector comprising an expression cassette, said cassette comprising a regulatory element and a nucleotide sequence operably linked to the regulatory element, wherein the regulatory element is capable of initiating cell-division-preferred and/or proliferation-preferred transcription of the nucleotide sequence in a plant cell, wherein the regulatory element comprises a nucleotide sequence selected from the group consisting of:
a) native sequences operably linked to DNA coding for maize PCNA2;
b) the nucleotide sequence set forth in SEQ ID NO: 1;
c) the nucleotide sequence set forth in SEQ ID NO: 2, positions 861 through 1276; and
d) the nucleotide sequence set forth in SEQ ID NO: 2, positions 331 through 1230.

15. The method of claim 14 further comprising regenerating a stably transformed plant from said transformed plant cell, wherein expression of said nucleotide sequence alters the phenotype of said plant tissue.

16. A plant cell stably transformed with an expression cassette comprising a regulatory element and a first nucleotide sequence operably linked to the regulatory element, wherein the regulatory element is capable of initiating cell-division-preferred and/or proliferation-preferred transcription of the nucleotide sequence in a plant cell, wherein the regulatory element comprises a nucleotide sequence selected from the group consisting of:
a) native sequences operably linked to DNA coding for maize PCNA2;
b) the nucleotide sequence set forth in SEQ ID NO: 1;
c) the nucleotide sequence set forth in SEQ ID NO: 2, positions 861 through 1276; and
d) the nucleotide sequence set forth in SEQ ID NO: 2, positions 331 through 1230.

17. The plant cell of claim 16, wherein said plant cell is maize, wheat, rice, barley, sorghum, or rye.


SEQUENCE LISTING (5 sequences)

SEQ ID NO: 1 - DNA, 900 bp, Zea mays
atcgtaatcggttttcaccgtataccgaaccgaaaaaaccgaataccaaactttatcaattcccaaatttgactattcgattatgtgaactaattgtgtgatacaattaaattgttattcacttatttgtatgtgatgtatgatgtatatctaaatatttgtacctatataatttttactttttaaaattatatgtaatctatcatgtaaacttgttgtatgtattgtcttgattataagtttggtattcggtttttaccgaaaaatcgaagtaaaaaaccgaaaccgaacttctcggtttttcattttctagaaaaccgaacggtttctaatgtttgaaaaaccgaagttttttaaaaccgaaaaaccgaaccgaagtttagaaaaaaaccgaatgcccagccctaaaaattagtaccccataagaactaaaaaaagataaaatgactaaaaattaatcagttgaaaccaaacctattttcccccacacctcacggtattgtttcgcattccaagtttgaaacacgactggaaacaaaacccaaaacgactggagggaccgagcttgtgctgagcagcagagatggcgggaaatgctgcgtctcccgcctcagtttcggatgccccgccctttcccaaaccggccaccgccgccgcccgtgtctccccaccgacaggtgggtccaatccttaaccacggaccagggcccccacctgtcaggtggaccttccgaagcaaggatcggccaggcgggaaaacatttcgcggcaggtggcggttgcgccaaatttctccctcccttttccgttcggcgtccccaaacgcctccctattaatctccccgcgttccccttccctcgcgccgccgctctcccctcccaaagctcgccccgctccca

SEQ ID NO: 2 - DNA, 4844 bp, Artificial Sequence
  Chemically synthesized plasmid
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcgcccttactatagggcacgcgtggtcgacggcccgggctggtatcgtaatcggttttcaccgtataccgaaccgaaaaaaccgaataccaaactttatcaattcccaaatttgactattcgattatgtgaactaattgtgtgatacaattaaattgttattcacttatttgtatgtgatgtatgatgtatatctaaatatttgtacctatataatttttactttttaaaattatatgtaatctatcatgtaaacttgttgtatgtattgtcttgattataagtttggtattcggtttttaccgaaaaatcgaagtaaaaaaccgaaaccgaacttctcggtttttcattttctagaaaaccgaacggtttctaatgtttgaaaaaccgaagttttttaaaaccgaaaaaccgaaccgaagtttagaaaaaaaccgaatgcccagccctaaaaattagtaccccataagaactaaaaaaagataaaatgactaaaaattaatcagttgaaaccaaacctattttcccccacacctcacggtattgtttcgcattccaagtttgaaacacgactggaaacaaaacccaaaacgactggagggaccgagcttgtgctgagcagcagagatggcgggaaatgctgcgtctcccgcctcagtttcggatgccccgccctttcccaaaccggccaccgccgccgcccgtgtctccccaccgacaggtgggtccaatccttaaccacggaccagggcccccacctgtcaggtggaccttccgaagcaaggatcggccaggcgggaaaacatttcgcggcaggtggcggttgcgccaaatttctccctcccttttccgttcggcgtccccaaacgcctccctattaatctccccgcgttccccttccctcgcgccgccgctctcccctcccaaagctcgccccgctcccaaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaattcagggcgcaagggctgctaaaggaaccggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcgccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgatccatggcgatgcctgcttgccgaatatcatggtggaaaatggccgcttttctggattcaacgactgtggccggctgggtgtggcggaccgctatcaggacatagcgttggatacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 3 - DNA, 427 bp, Oryza sativa
gaaaaataattcaaaatcattggagggaaatggccaagctgtgcggtcagcgagatggcgcgaaaggcaattccatctcccgcccagctcccagaacacccgcccttttccaaatcaccaagaccaagcacccgtcatccacttttcccccactgccaggtgggcccgtactccaccatggtcccacatgtcagctgaatcttccactgcaagaagcgggaaacatttcgggtggaggcgggtttccccccaatttctccctccatttttttttcgtctcgcgtcccccaaacccctccctattaatctcccccttccccctttcttcccctcgccgccgcctcctcctcccaaaccccacaagcgaatcctagggtttctcgcctcgccgccgcatcgccgccgggaagtctccgccgccgccatg

SEQ ID NO: 4 - DNA, 4844 bp, Artificial Sequence
  Chemically synthesized. Figure 3I - 3A, lower strand of figure
tcgcgggttatgcgtttggcggagaggggcgcgcaaccggctaagtaattacgtcgaccgtgctgtccaaagggctgacctttcgcccgtcactcgcgttgcgttaattacactcaatcgagtgagtaatccgtggggtccgaaatgtgaaatacgaaggccgagcatacaacacaccttaacactcgcctattgttaaagtgtgtcctttgtcgatactggtactaatgcggttcgaaccatggctcgagcctaggtgatcattgccggcggtcacacgaccttaagcgggaatgatatcccgtgcgcaccagctgccgggcccgaccatagcattagccaaaagtggcatatggcttggcttttttggcttatggtttgaaatagttaagggtttaaactgataagctaatacacttgattaacacactatgttaatttaacaataagtgaataaacatacactacatactacatatagatttataaacatggatatattaaaaatgaaaaattttaatatacattagatagtacatttgaacaacatacataacagaactaatattcaaaccataagccaaaaatggctttttagcttcattttttggctttggcttgaagagccaaaaagtaaaagatcttttggcttgccaaagattacaaactttttggcttcaaaaaattttggctttttggcttggcttcaaatctttttttggcttacgggtcgggatttttaatcatggggtattcttgatttttttctattttactgatttttaattagtcaactttggtttggataaaagggggtgtggagtgccataacaaagcgtaaggttcaaactttgtgctgacctttgttttgggttttgctgacctccctggctcgaacacgactcgtcgtctctaccgccctttacgacgcagagggcggagtcaaagcctacggggcgggaaagggtttggccggtggcggcggcgggcacagaggggtggctgtccacccaggttaggaattggtgcctggtcccgggggtggacagtccacctggaaggcttcgttcctagccggtccgcccttttgtaaagcgccgtccaccgccaacgcggtttaaagagggagggaaaaggcaagccgcaggggtttgcggagggataattagaggggcgcaaggggaagggagcgcggcggcgagaggggagggtttcgagcggggcgagggtttcccgcttaagacgtctataggtagtgtgaccgccggcgagctcgtacgtagatctcccgggttaagcgggatatcactcagcataatgttaagtgaccggcagcaaaatgttgcagcactgacccttttgggaccgcaatgggttgaattagcggaacgtcgtgtagggggaaagcggtcgaccgcattatcgcttctccgggcgtggctagcgggaagggttgtcaacgcgtcggacttaccgcttaccctgcgcgggacatcgccgcgtaattcgcgccgcccacaccaccaatgcgcgtcgcactggcgatgtgaacggtcgcgggatcgcgggcgaggaaagcgaaagaagggaaggaaagagcggtgcaagcggccgaaaggggcagttcgagatttagcccccgagggaaatcccaaggctaaatctcgaaatgccgtggagctggcgttttttgaactaaacccactaccaagtgcatcacccggtagcgggactatctgccaaaaagcgggaaactgcaacctcaggtgcaagaaattatcacctgagaacaaggtttgaccttgttgtgagttgggatagcgccagataagaaaactaaatattccctaaaacggctaaagccggataaccaattttttactcgactaaattgtttaagtcccgcgttcccgacgatttccttggccttgtgcatctttcggtcaggcgtctttgccacgactggggcctacttacagtcgatgacccgatagacctgttcccttttgcgttcgcgtttctctttcgtccatcgaacgtcacccgaatgtaccgctatcgatctgacccgccaaaatacctgtcgttcgcttggccttaacggtcgaccccgcgggagaccattccaacccttcgggacgtttcatttgacctaccgaaagaacggcggttcctagactaccgcgtcccctagttctagactagttctctgtcctactcctagcaaagcgtactaacttgttctacctaacgtgcgtccaagaggccggcgaacccacctctccgataagccgatactgacccgtgttgtctgttagccgacgagactacggcggcacaaggccgacagtcgcgtccccgcgggccaagaaaaacagttctggctggacaggccacgggacttacttgacgtcctgctccgtcgcgccgatagcaccgaccggtgctgcccgcaaggaacgcgtcgacacgagctgcaacagtgacttcgcccttccctgaccgacgataacccgcttcacggccccgtcctagaggacagtagagcggaacgaggacggctctttcataggtagtaccgactacgttacgccgccgacgtatgcgaactaggccgatggacgggtaagctggtggttcgctttgtagcgtagctcgctcgtgcatgagcctaccttcggccagaacagctagtcctactagacctgcttctcgtagtccccgagcgcggtcggcttgacaagcggtccgagttccgcgcgtacgggctgccgctcctagagcagcactaggtaccgctacggacgaacggcttatagtaccaccttttaccggcgaaaagacctaagttgctgacaccggccgacccacaccgcctggcgatagtcctgtatcgcaacctatgggcactataacgacttctcgaaccgccgcttacccgactggcgaaggagcacgaaatgccatagcggcgagggctaagcgtcgcgtagcggaagatagcggaagaactgctcaagaagacttaactttttccttctcatactcataagttgtaaaggcacagcgggaataagggaaaaaacgccgtaaaacggaaggacaaaaacgagtgggtctttgcgaccactttcattttctacgacttctagtcaacccacgtgctcacccaatgtagcttgacctagagttgtcgccattctaggaactctcaaaagcggggcttcttgcaaaaggttactactcgtgaaaatttcaagacgatacagtatgtgataatagggcataactgcggcccgttctcgttgagccagcggcccgcgccataagagtcttactgaaccaactcatgagtggtcagtgtcttttcgtagaatgcctaccgtactgtcattctcttaatacgtcacgacggtattggtactcactattgtgacgccggttgaatgaagactgttgctagcctcctggcttcctcgattggcgaaaaaacgtgttgtaccccctagtacattgagcggaactagcaacccttggcctcgacttacttcggtatggtttgctgctctcactgtggtgctacggacatcgttacggttgttgcaacgcgtttgataattgaccgcttgatgaatgagatcgaagggccgttgttaattatctgacctacctccgcctatttcaacgtcctggtgaagacgcgagccgggaaggccgaccgaccaaataacgactatttagacctcggccactcgcacccagagcgccatagtaacgtcgtgaccccggtctaccattcgggagggcatagcatcaatagatgtgctgcccctcagtccgttgatacctacttgctttatctgtctagcgactctatccacggagtgactaattcgtaaccattgacagtctggttcaaatgagtatatatgaaatctaactaaattttgaagtaaaaattaaattttcctagatccacttctaggaaaaactattagagtactggttttagggaattgcactcaaaagcaaggtgactcgcagtctggggcatcttttctagtttcctagaagaactctaggaaaaaaagacgcgcattagacgacgaacgtttgtttttttggtggcgatggtcgccaccaaacaaacggcctagttctcgatggttgagaaaaaggcttccattgaccgaagtcgtctcgcgtctatggtttatgacaggaagatcacatcggcatcaatccggtggtgaagttcttgagacatcgtggcggatgtatggagcgagacgattaggacaatggtcaccgacgacggtcaccgctattcagcacagaatggcccaacctgagttctgctatcaatggcctattccgcgtcgccagcccgacttgccccccaagcacgtgtgtcgggtcgaacctcgcttgctggatgtggcttgactctatggatgtcgcactcgtaactctttcgcggtgcgaagggcttccctctttccgcctgtccataggccattcgccgtcccagccttgtcctctcgcgtgctccctcgaaggtccccctttgcggaccatagaaatatcaggacagcccaaagcggtggagactgaactcgcagctaaaaacactacgagcagtccccccgcctcggatacctttttgcggtcgttgcgccggaaaaatgccaaggaccggaaaacgaccggaaaacgagtgtacaagaaaggacgcaataggggactaagacacctattggcataatggcggaaactcactcgactatggcgagcggcgtcggcttgctggctcgcgtcgctcagtcactcgctccttcgccttc

SEQ ID NO: 5 - DNA, 4844 bp, Artificial Sequence; identical to SEQ ID NO: 2
  Chemically synthesized. Figure 3I - 3A, upper strand of figure